(19) United States
(12) Patent Application Publication
(10) Pub. No.: US 2021/0279918 A1
(43) Pub. Date: Sep. 9, 2021

(54) MEDICAL INFORMATION PROCESSING APPARATUS, MEDICAL IMAGE DIAGNOSIS APPARATUS, AND MEDICAL INFORMATION PROCESSING METHOD

(71) Applicant: CANON MEDICAL SYSTEMS CORPORATION, Otawara-shi (JP)

(72) Inventors: Kusuto KOGA, Nasushiobara (JP); Kazuki GATAYAMA, Otawara (JP); Katsuhiko ISHIDA, Nasushiobara (JP); Tadatsugu NUNOME, Otawara (JP); Ryusei SAIKI, Otawara (JP)

(73) Assignee: CANON MEDICAL SYSTEMS CORPORATION, Otawara-shi (JP)

(21) Appl. No.: 17/188,770

(22) Filed: Mar. 1, 2021

(30) Foreign Application Priority Data
Mar. 3, 2020 (JP) 2020-036082

(51) Int. Cl. G06T 11/00; A61B 5/00; G06T 7/00; G06T 5/50

(52) U.S. Cl. CPC G06T 11/006; A61B 5/0035; G06T 7/0012; G06T 2211/421; G06T 2211/408; G06T 2207/20224; G06T 2207/10081; G06T 5/50

(57) ABSTRACT

A medical information processing apparatus according to an embodiment includes a processing circuit. The processing circuit is configured: to generate, on the basis of first data obtained in a first imaging process, second data equivalent to data obtained in an imaging process performed under an image taking condition different from that of the first imaging process; to generate, on the basis of quality of the second data, assistance information to assist reviewing related to a second imaging process scheduled to be executed later than the first imaging process under an image taking condition different from that of the first imaging process; and to cause a display circuit to display the assistance information prior to the execution of the second imaging process.

### CROSS-REFERENCE TO RELATED APPLICATIONS

This application is based upon and claims the benefit of priority from Japanese Patent Application No. 2020-036082, filed on Mar. 3, 2020; the entire contents of which are incorporated herein by reference.

### FIELD

Embodiments described herein relate generally to a medical information processing apparatus, a medical image diagnosis apparatus, and a medical information processing method.

### BACKGROUND

Normally, during imaging processes using an X-ray computed tomography apparatus (an X-Ray CT apparatus), a position determining image is at first obtained through an imaging process using a relatively low radiation dose (hereinafter “dose”) called a position determining imaging process, before performing an imaging process (a main imaging process) for the purpose of obtaining a diagnosis image, on an imaged region set by using the position determining image, while using a higher dose than that of the position determining imaging process. In recent years, there are some X-ray CT apparatuses configured to obtain three-dimensional data in the position determining imaging process.

The imaging processes using X-ray CT apparatuses as described above are executed according to imaging protocols that are set prior to the imaging processes. In the settings of an imaging protocol, for example, an imaged site, an image taking condition for the position determining imaging process or the main imaging process, an imaged range, a reconstruction condition, and the like are set. When the imaging process is started according to the imaging protocol, various types of processes included in the imaging protocol are performed according to the image taking condition and the like in the settings.

Further, methods have been developed to enhance the image quality and the definition of projection data and reconstructed images, by using artificial intelligence such as a deep learning model.

Regarding image diagnosis processes using an X-ray CT apparatus or the like, no technique has so far been established by which an imaging protocol being once set can be verified using an objective index so as to review a workflow related to the image diagnosis processes.

### BRIEF DESCRIPTION OF THE DRAWINGS

FIG. 1 is a block diagram illustrating an exemplary configuration of an X-ray computed tomography apparatus in which a medical information processing apparatus according to an embodiment is incorporated;

FIG. 2 is a chart for explaining an example of a data generating process performed by a data generating function 150d included in a processing circuit 150 in which low dose projection data is used as an input, whereas high dose equivalent projection data is used as an output;

FIG. 3 is a chart for explaining another example of the data generating process performed by the data generating function 150d included in the processing circuit 150 in which low dose projection data is used as an input, whereas high dose equivalent reconstructed image data is used as an output;

FIG. 4 is a chart for explaining yet another example of the data generating process performed by the data generating function 150d included in the processing circuit 150 in which a low dose reconstructed image is used as an input, whereas high dose equivalent reconstructed image data is used as an output;

FIG. 5 is a flowchart illustrating an example of a flow in an evaluating process and an assistance information generating process;

FIG. 6 is a drawing for explaining an example of the process of generating a high dose equivalent image at step S3 in which high dose equivalent projection data is generated by inputting low dose projection data so that high dose equivalent reconstructed data is output;

FIG. 7 is a drawing for explaining another example of the process of generating a high dose equivalent image at step S3 in which low dose reconstructed data is generated by inputting low dose projection data so that high dose equivalent reconstructed data is output; and

FIG. 8 is a drawing illustrating an example of displaying assistance information, a high dose equivalent image, and the like on a display device 42.

### DETAILED DESCRIPTION

A medical information processing apparatus according to an embodiment includes a processing circuit. The processing circuit is configured: to generate, on the basis of first data obtained in a first imaging process, second data equivalent to data obtained in an imaging process performed under an image taking condition different from that of the first imaging process; to generate, on the basis of quality of the second data, assistance information to assist reviewing related to a second imaging process scheduled to be executed later than the first imaging process under an image taking condition different from that of the first imaging process; and to cause a display circuit to display the assistance information prior to the execution of the second imaging process.

Exemplary embodiments of a medical information processing apparatus, a medical information processing method, and a medical information processing program will be explained in detail below, with reference to the accompanying drawings.

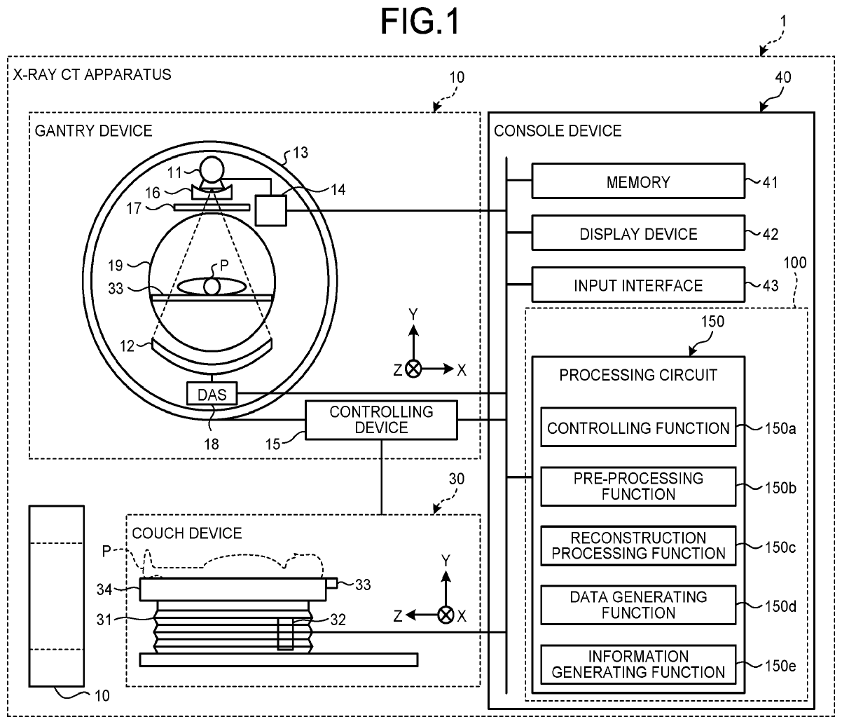

FIG. 1 is a block diagram illustrating an exemplary configuration of an X-ray Computed Tomography apparatus 1 (hereinafter, “X-ray CT apparatus 1”) in which a medical information processing apparatus 100 according to an embodiment is incorporated. As illustrated in FIG. 1, the X-ray CT apparatus 1 includes a gantry device 10, a couch device 30, and a console device 40.

In the present embodiment, the rotation axis of a rotating frame 13 in a non-tilt state or the longitudinal direction of a couchtop 33 of the couch device 30 is defined as a Z-axis direction; an axial direction orthogonal to the Z-axis direction and parallel to the floor surface is defined as an X-axis direction; and an axial direction orthogonal to the Z-axis direction and perpendicular to the floor surface is defined as a Y-axis direction.

The gantry device 10 includes an imaging system for taking medical images used for diagnosis purposes. In other words, the gantry device 10 is a device including the imaging system configured to radiate X-rays onto an examined subject (hereinafter “patient”) P and to acquire projection data from detection data of X-rays that have passed through the patient P. The gantry device 10 includes an X-ray tube 11, a wedge 16, a collimator 17, an X-ray detector 12, an X-ray high-voltage device 14, a slip ring 19, a Data Acquisition System (DAS) 18, the rotating frame 13, a controlling device 15, and the couch device 30.

The X-ray tube 11 is a vacuum tube configured to emit thermo electrons from a negative pole (a filament) toward a positive pole (a target), with high voltage applied from the X-ray high-voltage device 14.

The wedge 16 is a filter for adjusting the X-ray amount of the X-rays radiated from the X-ray tube 11. More specifically, the wedge 16 is a filter configured to pass and attenuate the X-rays radiated from the X-ray tube 11, so that the X-rays radiated from the X-ray tube 11 onto the patient P have a predetermined distribution.

The wedge 16 is, for example, a wedge filter or a bow-tie filter and is a filter obtained by processing aluminum so as to have a predetermined target angle and a predetermined thickness.

The collimator 17 is configured with lead plates or the like used for narrowing down the radiated range of the X-rays that have passed through the wedge 16 and is configured to form a slit with a combination of the plurality of lead plates or the like.

The X-ray detector 12 is configured to detect the X-rays that were radiated from the X-ray tube 11 and have passed through the patient P and to output an electrical signal corresponding to the X-ray amount to the data acquisition device (DAS 18). For example, the X-ray detector 12 includes a plurality of rows of X-ray detecting elements in each of which a plurality of X-ray detecting elements are arranged in a channel direction along an arc centered on a focal point of the X-ray tube 11. For example, the X-ray detector 12 includes a plurality of rows of X-ray detecting elements in each of which a plurality of X-ray detecting elements are arranged in a channel direction along an arc centered on a focal point of the X-ray tube. For example, the X-ray detector 12 has a structure in which the plurality of rows of X-ray detecting elements are arranged in a slice direction (which may be called a body axis direction or a row direction), the plurality of rows each having the plurality of X-ray detecting elements arranged in the channel direction.

Further, for example, the X-ray detector 12 is a detector of an indirect conversion type and includes a grid, a scintillator array, and an optical sensor array. The scintillator array includes a plurality of scintillators each including a scintillator crystal that outputs light in a photon quantity corresponding to an X-ray amount becoming incident thereto. The grid is arranged on a surface of the scintillator array that is positioned on the X-ray incident side and includes an X-ray blocking plate having a function of absorbing scattered X-rays. The optical sensor array has a function of converting the light amounts from the scintillators into corresponding electrical signals and includes optical sensors configured with Photomultiplier Tubes (PMTs), for example. Alternatively, the X-ray detector 12 may be a detector of a direct conversion type that includes a semiconductor element configured to convert X-rays becoming incident thereto into an electrical signal.

The X-ray high-voltage device 14 includes: a high-voltage generating device including electrical circuits such as a transformer, a rectifier, and the like and having a function of generating the high voltage to be applied to the X-ray tube 11; and an X-ray controlling device configured to control the output voltage in accordance with the X-rays radiated by the X-ray tube 11. The high-voltage generating device may be of a transformer type or of an inverter type. Further, the X-ray high-voltage device 14 may be provided on the rotating frame 13 or may be provided so as to belong to a fixed frame (not illustrated) of the gantry device 10. The fixed frame is a frame configured to rotatably support the rotating frame 13.

The DAS 18 includes an amplifier configured to perform an amplifying process on the electrical signals output from the X-ray detecting elements of the X-ray detector 12 and an Analog/Digital (A/D) converter configured to convert the electrical signals into digital signals. The DAS 18 is configured to generate the detection data. The detection data generated by the DAS 18 is transferred to the console device 40.

The rotating frame 13 is an annular frame configured to support the X-ray tube 11 and the X-ray detector 12 so as to oppose each other and configured to rotate the X-ray tube 11 and the X-ray detector 12 via the controlling device 15. In addition to supporting the X-ray tube 11 and the X-ray detector 12, the rotating frame 13 may further support the X-ray high-voltage device 14 and/or the DAS 18. Further, the detection data generated by the DAS 18 is, in an example, transmitted from a transmitter including a light emitting diode and being provided on the rotating frame 13, to a receiver including a photodiode and being provided in a non-rotation part (e.g., the fixed frame) of the gantry device 10, through optical communication, and is further transferred to the console device 40. The method for transmitting the detection data from the rotating frame 13 to the non-rotation part of the gantry device 10 is not limited to optical communication and may be realized with any of other contactless data transfer methods.

The controlling device 15 includes: a processing circuit having a Central Processing Unit (CPU) or the like; and a driving mechanism configured with a motor, an actuator, and/or the like. Upon receipt of an input signal from an input interface 43 attached to the console device 40 or from an input interface attached to the gantry device 10, the controlling device 15 has a function of controlling operations of the gantry device 10 and the couch device 30. Further, upon receipt of input signals, the controlling device 15 is configured to exercise control so as to rotate the rotating frame 13 and to bring the gantry device 10 and the couch device 30 into operation.

For example, the controlling device 15 is configured to tilt the gantry device 10, as a result of the controlling device 15 rotating the rotating frame 13 on an axis parallel to the X-axis direction, on the basis of tilting angle (tilt angle) information input thereto by the input interface attached to the gantry device 10. The controlling device 15 and a controlling function 150a included in the processing circuit 150 are examples of the controlling unit.

The couch device 30 is a device on which the patient P to be scanned is placed and which is configured to move the patient P. The couch device 30 includes a base 31, a couch driving device 32, the couchtop 33, and a supporting frame 34. The base 31 is a casing configured to support the supporting frame 34 so as to be movable in the vertical directions. The couch driving device 32 is a motor or an actuator configured to move the couchtop 33 on which the patient P is placed, along the long axis directions thereof (the Z-axis directions in FIG. 1). The couchtop 33 provided on the top face of the supporting frame 34 is a board on which the patient P is placed. In addition to the couchtop 33, the couch driving device 32 may also move the supporting frame 34 along the long axis directions of the couchtop 33.

The couch driving device 32 is configured to move the base 31 in up-and-down directions, according to control signals from the controlling device 15. The couch driving device 32 is configured to move the couchtop 33 in the long axis directions according to control signals from the controlling device 15.

The console device 40 is a device configured to receive operations performed on the X-ray CT apparatus 1 by a user and to reconstruct X-ray CT image data from X-ray detection data acquired by the gantry device 10. The console device 40 includes a memory 41, a display device 42, the input interface 43, and the processing circuit 150.

The medical information processing apparatus 100 according to the present embodiment is realized with at least the processing circuit 150. The medical information processing apparatus 100 according to the present embodiment may further include the memory 41, the display device 42, and the input interface 43, for example.

The memory 41 is realized by using, for example, a semiconductor memory element such as a Random Access Memory (RAM) or a flash memory, or a hard disk, an optical disk, or the like. For example, the memory 41 is configured to store therein the projection data and reconstructed image data. The memory 41 is an example of a storage unit.

Further, the memory 41 has stored therein dedicated computer programs (hereinafter “programs”) for realizing the controlling function 150a, a pre-processing function 150b, a reconstruction processing function 150c, a data generating function 150d, and an information generating function 150e described later.

The display device 42 is a monitor referenced by the user and is configured to display various types of information. For example, the display device 42 is configured to output medical images (CT images) generated by the processing circuit 150, a Graphical User Interface (GUI) used for receiving various types of operations from the user, and the like. For example, the display device 42 is a liquid crystal display device or a Cathode Ray Tube (CRT) display device. The display device 42 is an example of a display unit.

The input interface 43 is configured to receive various types of input operations from the user, to convert the received input operations into electrical signals, and to output the electrical signals to the processing circuit 150. For example, the input interface 43 is configured to receive, from the user, an acquisition condition used at the time of acquiring the projection data, a reconstruction condition used at the time of reconstructing a CT image, an image processing condition used at the time of generating a post-processing image from the CT image, and the like. Further, for example, the input interface 43 is realized by using a mouse, a keyboard, a trackball, a switch, a button, a joystick, and/or the like. The input interface 43 is an example of an input unit.

The processing circuit 150 is configured to control operations of the entirety of the X-ray CT apparatus 1. For example, the processing circuit 150 includes the controlling function 150a, the pre-processing function 150b, the reconstruction processing function 150c, the data generating function 150d, and the information generating function 150e. In the embodiment, processing functions executed by the constituent elements, namely, the controlling function 150a, the pre-processing function 150b, the reconstruction processing function 150c, the data generating function 150d, and the information generating function 150e, are stored in the memory 41 in the form of computer-executable programs. The processing circuit 150 is a processor configured to realize the functions corresponding to the programs by reading and executing the programs from the memory 41. In other words, the processing circuit 150 that has read the programs has the functions illustrated within the processing circuit 150 in FIG. 1.

With reference to FIG. 1, an example was explained in which the single processing circuit (i.e., the processing circuit 150) realizes the processing functions executed by the controlling function 150a, the pre-processing function 150b, the reconstruction processing function 150c, the data generating function 150d, and the information generating function 150e. However, it is also acceptable to structure the processing circuit 150 by combining together a plurality of independent processors, so that the functions are realized as a result of the processors executing the programs.

In other words, each of the abovementioned functions may be structured as a program, so that a single processing circuit executes the programs. Alternatively, one or more specific functions may be installed in a dedicated and independent program executing circuit.

The term “processor” used in the above explanations denotes, for example, a Central Processing Unit (CPU), a Graphical Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The one or more processors realize the functions by reading and executing the programs saved in the memory 41. Further, instead of saving the programs in the memory 41, it is also acceptable to directly incorporate the programs into the circuits of the processors. In that situation, the processors realize the functions by reading and executing the programs incorporated in the circuits thereof.

By employing the controlling function 150a, the processing circuit 150 is configured to control various types of functions of the processing circuit 150, on the basis of input operations received from the user via the input interface 43. Further, by employing the controlling function 150a, the processing circuit 150 is configured to cause the display device 42 to display a high dose equivalent image and assistance information. Further, by employing the controlling function 150a, the processing circuit 150 is configured to cause the display device 42 to display a measurement tool for setting a region of interest and at least one GUI for exercising control based on the assistance information.

By employing the pre-processing function 150b, the processing circuit 150 is configured to generate data obtained by performing pre-processing processes such as a logarithmic conversion process, an offset process, an inter-channel sensitivity correcting process, a beam hardening correction, and/or the like on the detection data output from the DAS 18. The data (the detection data) before the pre-processing processes and the data after the pre-processing processes may collectively be referred to as projection data. By employing the reconstruction processing function 150c, the processing circuit 150 is configured to generate the CT image data by performing a reconstructing process using a filtered back projection method, a successive approximation reconstruction method, and/or the like on the projection data generated by the pre-processing function 150b. Further, by employing the reconstruction processing function 150c, the processing circuit 150 is configured to convert the CT image data resulting from the reconstruction into tomographic image data on an arbitrary cross-sectional plane or three-dimensional image data by using a publicly-known method, on the basis of an input operation received from the user via the input interface 43.

By employing the data generating function 150d, the processing circuit 150 is configured, on the basis of first data (e.g., a position determining image) obtained in a first imaging process (e.g., a position determining imaging process or a main imaging process serving as a preliminary imaging process), to generate second data equivalent to data obtained in an imaging process performed under an image taking condition different from that of the first imaging process. In this situation, for X-ray CT apparatuses, the image taking condition being different denotes, for example, that the X-ray tube voltage or the X-ray tube current used at the time of radiating the X-rays is different. Further, for Magnetic Resonance Imaging (MRI) apparatuses, the image taking condition being different denotes, for example, that the intensity or the frequency of electromagnetic pulses used for the imaging process is different.

Further, for X-ray CT apparatuses, the imaging process performed under the image taking condition different from that of the first imaging process denotes, for example, a main imaging process scheduled to be performed later than a position determining imaging process serving as the first imaging process or a main imaging process scheduled to be performed later than another main imaging process serving as the first imaging process. Further, for MRI apparatuses, the imaging process performed under the image taking condition different from that of the first imaging process denotes, for example, at least one of multiple sessions of a main imaging process performed while varying the image taking condition little by little, after performing the first imaging process represented by a main imaging process.

Further, the imaging process performed under the image taking condition different from that of the first imaging process may be referred to as a second imaging process. Also, the second data is equivalent to data being more suitable for diagnosis processes than the first data is (and is typically equivalent to data obtained in the second imaging process). However, the second data does not necessarily have to be equivalent to data obtained in the second imaging process.

Further, the main imaging process denotes an imaging process performed with a relatively high dose, for the purpose of obtaining an image (a diagnosis image) used for performing a diagnosis process on a diagnosis target. Further, the position determining imaging process denotes an imaging process performed with a lower dose than in the main imaging process, for the purpose of determining an imaged range of the main imaging process. Further, an imaging protocol denotes information including a diagnosed site, an image taking condition for a position determining imaging process or a main imaging process, a contrast enhancing method, a reconstruction condition, an image display method, and/or the like. Further, in the present embodiment, it is assumed that, through the main imaging process or the position determining imaging process, data (volume data) corresponding to a three-dimensional region of the patient is obtained.

In the present embodiment, the data generating function 150d of the processing circuit 150 includes an Artificial Intelligence (AI) model of which a typical example is a deep neural network.

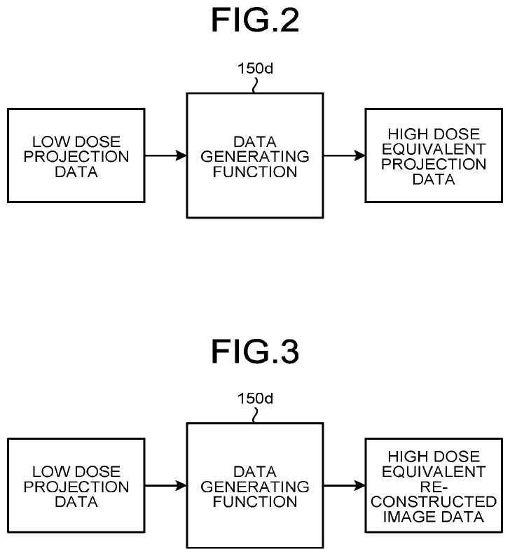

FIGS. 2, 3, and 4 are charts for explaining data generating processes performed by the data generating function 150d included in the processing circuit 150.

As illustrated in FIG. 2, by employing the data generating function 150d, the processing circuit 150 receives an input of projection data (which hereinafter may be referred to as “low dose projection data”) obtained in a position determining imaging process, for example, and generates and outputs projection data (hereinafter, “high dose equivalent projection data”) having image quality equivalent to that of projection data obtained in a main imaging process. In this situation, it is possible to generate the data generating function 150d of the processing circuit 150 serving as the AI model and illustrated in FIG. 2, through a learning process that uses training-purpose data in which, for example, low dose projection data is used as an input, whereas high dose equivalent projection data is used as training data.

Further, as illustrated in FIG. 3, by employing the data generating function 150d, the processing circuit 150 receives an input of low dose projection data, for example, and generates and outputs reconstructed image data (hereinafter, “high dose equivalent reconstructed image data”) having image quality equivalent to that of reconstructed image data reconstructed by using projection data obtained in a main imaging process. In this situation, it is possible to generate the data generating function 150d of the processing circuit 150 serving as the AI model and illustrated in FIG. 3, through a learning process that uses training-purpose data in which, for example, low dose projection data is used as an input, whereas high dose equivalent reconstructed image data is used as training data.

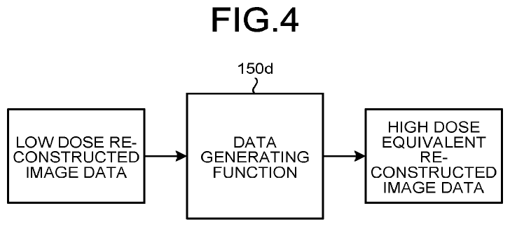

Further, as illustrated in FIG. 4, by employing the data generating function 150d, the processing circuit 150 receives an input of reconstructed image data (hereinafter, “low dose reconstructed image data”) obtained in a reconstructing process using low dose projection data, for example, and generates and outputs high dose equivalent reconstructed image data. In this situation, it is possible to generate the data generating function 150d of the processing circuit 150 serving as the AI model and illustrated in FIG. 4, through a learning process that uses training-purpose data in which, for example, low dose reconstructed image data is used as an input, whereas high dose reconstructed image data is used as training data.

By employing the information generating function 150e, on the basis of the quality of the second data (e.g., a position determining image or an image taken in a main imaging process), the processing circuit 150 is configured to generate assistance information to assist reviewing related to a second imaging process scheduled to be executed later than the first imaging process under an image taking condition different from that of the first imaging process. For example, by employing the information generating function 150e, the processing circuit 150 is configured to perform an evaluating process using one of the high dose equivalent projection data and the high dose equivalent reconstructed image data that was generated and to further generate the assistance information to assist reviewing of a workflow including an imaging protocol, on the basis of the evaluating process. The information generating function 150e in this situation is realized by using a rule-based computation function, an AI model, or a combination of an AI model and a rule-based computation function.

In this situation, the “assistance information to assist the reviewing related to the second imaging process to be performed under an image taking condition different from that of the first imaging process” denotes information used for reviewing the imaging protocol or reviewing related to reconstruction and post-processing processes with regard to the second imaging process scheduled to be executed later than the first imaging process. Further, the “evaluating process using one of the high dose equivalent projection data and the high dose equivalent reconstructed image data” denotes a process such as: calculating an evaluation value to evaluate the image quality of the high dose equivalent projection data, the high dose equivalent reconstructed image data, or an image (hereinafter “high dose equivalent image”) generated from one of the high dose equivalent projection data and the high dose equivalent reconstructed image data; detecting an abnormal site by using the high dose equivalent image; calculating a replacement level of the high dose equivalent image for a high dose image; and/or obtaining at least one of a main imaging process necessity judgment result and a main imaging process protocol suitability judgment result based on the evaluation value, the detection result, and the replacement level.

Further, the “assistance information based on the evaluating process” (hereinafter, simply “assistance information”) is information used for assisting optimization of a workflow in an image diagnosis process and includes at least one of: information (evaluation information) obtained in the evaluating process; the result of judging whether or not the main imaging process needs to be performed based on the evaluation information; a revision proposal for a protocol of the main imaging process based on the evaluation information; and a replacement proposal for a protocol of the main imaging process based on the evaluation information.

In the following sections, a number of specific examples will be explained with regard to the evaluating process and the assistance information generating process performed by the information generating function 150e.

For example, by employing the information generating function 150e, the processing circuit 150 is configured to make an evaluation by using a statistic value and an image quality evaluation mathematical function related to the high dose equivalent reconstructed image. For example, by employing the information generating function 150e, the processing circuit 150 calculates the statistic value and the image quality evaluation mathematical function, with regard to one (or more) region(s) of interest set in the high dose equivalent image. In this situation, the statistic value may be an SD value, for example. The image quality evaluation mathematical function may be, for example, a contrast ratio, a spatial resolution, a Modulation Transfer Function (MTF) value, or a Slice Sensitivity Function (SSP) value. Further, by employing the information generating function 150e, the processing circuit 150 judges whether or not the main imaging process needs to be performed and whether or not the protocol of the main imaging process is suitable on the basis of the statistic value and the image quality evaluation mathematical function serving as evaluation values and further generates the assistance information including the judgment results and the evaluation values serving as the basis thereof.

The region of interest may be set in the high dose equivalent image by performing a manual operation using a measurement tool (explained later). Alternatively, it is also acceptable to extract a site or an organ by using a region dividing process (segmentation) or an anatomical classification process, so as to automatically set the region of interest in accordance with diagnosis purposes.

Further, by employing the information generating function 150e, the processing circuit 150 is configured to receive an input of the high dose equivalent image and to output the statistic value and the image quality evaluation mathematical function with regard to the entire region or a partial region of the input image. In this situation, it is possible to realize an AI model including the information generating function 150e configured in this manner, through a learning process using training-purpose data in which, for example, high dose equivalent images are used as input data, whereas statistic values and image quality evaluation mathematical functions are used as training data. Further, the high dose equivalent images used as the input data may be input as the entire images, so that the AI model includes the process of setting a region of interest subject to the calculation of the statistic value and the image quality evaluation mathematical function. Alternatively, the AI model may use, as the input, pixels within a region of interest being set through a manual operation or an automatic operation.

Further, by employing the information generating function 150e, the processing circuit 150 is configured to receive an input of the high dose equivalent image and to detect whether or not an abnormal site such as a lesion is present. Upon detection of an abnormal site, the processing circuit 150 is configured, by employing the information generating function 150e, to judge whether or not an already-set imaging protocol is suitable for diagnosing the detected abnormal site (the imaging protocol suitability judgment). When determining that the already-set imaging protocol is not suitable, the processing circuit 150 is configured, by employing the information generating function 150e, to generate the assistance information including a revision proposal for the imaging protocol required to closely inspect the detected abnormal site and/or a replacement proposal for the imaging protocol.

In this situation, it is possible to realize an AI model configured to detect the abnormal site as described above, through a learning process using training-purpose data in which, for example, a plurality of images are used as input data, whereas abnormal site regions each being a detection result from a different one of the images are used as training data. In another example, it is possible to realize an AI model configured to judge whether or not the imaging protocol is suitable, through a learning process using training-purpose data in which a plurality of sets made up of information related to abnormal sites and imaging protocols are used as input data, whereas one or more imaging protocols required to closely inspect the corresponding abnormal sites are used as the training data. Alternatively, the imaging protocol suitability judgment does not necessarily have to be made by an AI model and may be made through a judging process using a table.

Further, by employing the information generating function 150e, the processing circuit 150 is configured to obtain a replaceability level of one of the high dose equivalent projection data and the high dose equivalent reconstructed image data for the high dose image. By employing the information generating function 150e, the processing circuit 150 is configured to judge whether or not it is appropriate to perform the main imaging process, on the basis of the obtained replaceability level.

It is possible to realize an AI model configured to obtain the replaceability level as described above, through a learning process using training-purpose data in which, for example, a plurality of sets made up of pieces of high dose equivalent projection data and pieces of high dose projection data or a plurality of sets made up of pieces of high dose equivalent reconstructed image data and pieces of high dose reconstructed image data are used as input data, whereas replaceability levels (e.g., five levels from levels 1 to 5) are used as training data. Alternatively, the process of judging whether or not it is appropriate to perform the main imaging process on the basis of the replaceability level may be realized with an AI model or through a judging process using a table, for example.

Further, by employing the information generating function 150e, the processing circuit 150 is configured to generate the assistance information including an image taking condition or a reconstruction condition recommended for the main imaging process, on the basis of one of the high dose equivalent projection data and the high dose equivalent reconstructed image data.

It is possible to realize an AI model configured to obtain the image taking condition or the reconstruction condition recommended for the main imaging process as described above, through a learning process using training-purpose data in which, for example, high dose equivalent projection data or high dose equivalent reconstructed image data and an SD value desired by the user are used as input data, whereas image taking conditions or reconstruction conditions required in the main imaging process to realize the SD values indicated in the input data are used as training data. In another example, it is possible to realize the AI model, through a learning process using training-purpose data in which, for example, a set made up of high dose equivalent data and high dose data (projection data or reconstructed image data actually obtained by using a high dose in the main imaging process) is used as input data, whereas image taking conditions or reconstruction conditions required in the main imaging process to realize the image quality of the high dose data indicated in the input data are used as training data. In addition, diagnosed sites may further be added to the input data.

Next, the evaluating process and the assistance information generating process performed by the medical information processing apparatus 100 according to the embodiment will be explained.

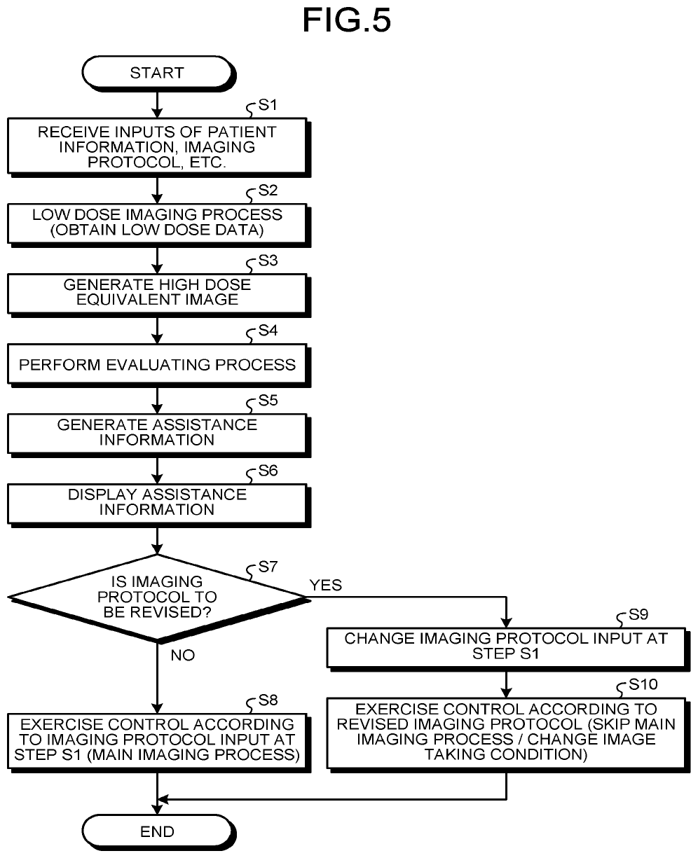

FIG. 5 is a flowchart illustrating a flow in the evaluating process and the assistance information generating process.

As illustrated in FIG. 5, at first, by employing the controlling function 150a, the processing circuit 150 receives inputs of inputs of patient information, an imaging protocol, and the like (step S1). In this situation, via the input interface 43, a diagnosed site (e.g., the head, the chest, etc.), image taking conditions of a position determining imaging process and a main imaging process, a contrast enhancement method, a reconstruction condition, an image display method, and the like are input as the imaging protocol.

Subsequently, by employing the controlling function 150a, the processing circuit 150 performs a position determining imaging process by using a low dose and obtains low dose projection data (step S2).

After that, by using the data generating function 150d, the processing circuit 150 generates a high dose equivalent image by using the low dose projection data (step S3).

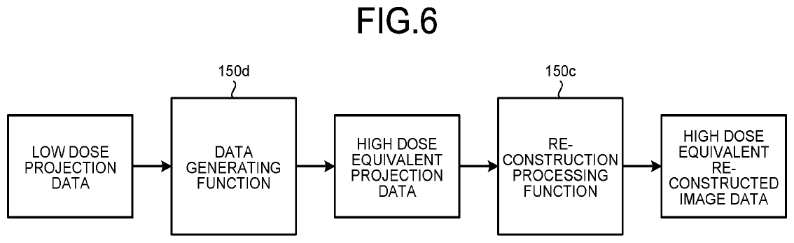

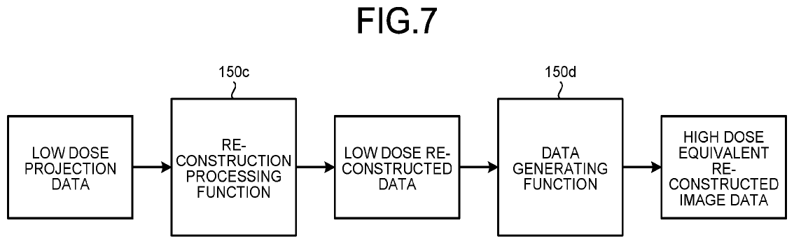

FIGS. 6 and 7 are drawing for explaining the process of generating the high dose equivalent image at step S3. As illustrated in FIG. 6, by employing the data generating function 150d, the processing circuit 150 receives the input of the low dose projection data and generates and outputs high dose equivalent projection data. By employing the reconstruction processing function 150c, the processing circuit 150 generates and outputs high dose equivalent reconstructed image data from the high dose equivalent projection data.

In another example, as illustrated in FIG. 7, by employing the reconstruction processing function 150c, the processing circuit 150 generates and outputs low dose equivalent reconstructed image data from the low dose projection data. By employing the data generating function 150d, the processing circuit 150 receives the input of the low dose equivalent reconstructed image data and generates and outputs high dose equivalent reconstructed image data.

In yet another example, as illustrated in FIG. 3, by employing the data generating function 150d, the processing circuit 150 receives the input of the low dose projection data and generates and outputs high dose equivalent reconstructed image data.

Subsequently, by employing the information generating function 150e, the processing circuit 150 performs the evaluating process (step S4). In the evaluating process, for example, one or more of the following processes are performed, as explained above: calculating the evaluation value of the image quality of the high dose equivalent image; detecting an abnormal site by using the high dose equivalent image; calculating the replacement level of the high dose equivalent image for the high dose image; and judging whether or not the main imaging process needs to be performed and whether or not the protocol of the main imaging process is suitable on the basis of the evaluation value, the detection result, and the replacement level.

After that, by employing the information generating function 150e, the processing circuit 150 generates assistance information based on the evaluating process (step S5). As a result of the assistance information generating process, the assistance information is generated, for example, so as to include one or more of the following as explained above: the evaluation information obtained in the evaluating process; the result of judging whether or not the main imaging process needs to be performed based on the evaluation information; a revision proposal for the protocol of the main imaging process based on the evaluation information; and a replacement proposal for the protocol of the main imaging process based on the evaluation information.

Next, by employing the controlling function 150a, the processing circuit 150 causes the display device 42 to display the high dose equivalent image, the assistance information, and the like (step S6).

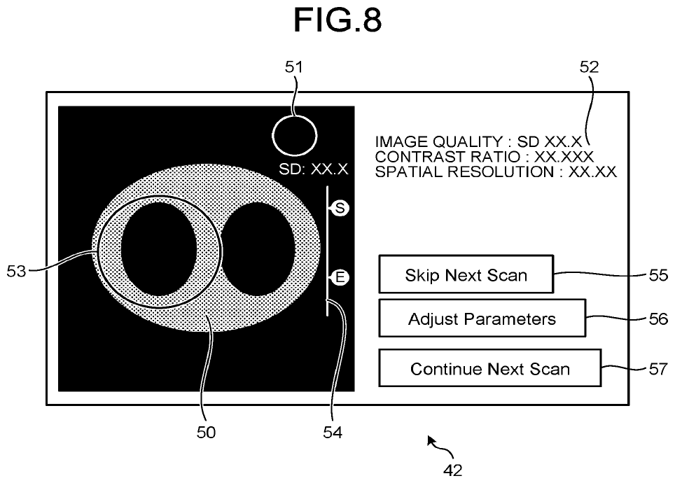

FIG. 8 is a drawing illustrating an example of displaying the assistance information, the high dose equivalent image, and the like on the display device 42. As illustrated in FIG. 8, the display device 42 displays assistance information 52 including a high dose equivalent image 50, an SD value, a contrast ratio, and a spatial resolution value. Further, it is also possible to display an image (a low dose reconstructed image) obtained by reconstructing the low dose projection data, in place of the high dose equivalent image or in a position next to the high dose equivalent image. By viewing the high dose equivalent image 50, the assistance information 52, and the like being displayed, the user is able to study and judge whether or not the main imaging process needs to be performed and whether or not the already-set imaging protocol is suitable.

Further, by employing the controlling function 150a, the processing circuit 150 is configured to cause the display device 42 to display, as necessary, a measurement tool 51 realized with a GUI for setting a region of interest. When manually setting a region of interest during the evaluating process, the user is able to set as many regions of interest as desired in desirable positions within the high dose equivalent image 50, by operating the measurement tool 51 via the input interface 43. Further, when the regions of interest have been set by using the measurement tool 51, the assistance information 52 corresponding to the regions of interest will automatically be set.

Further, by employing the controlling function 150a, the processing circuit 150 causes the display device 42 to display the following, as a GUI for exercising control based on the assistance information: imaged range changing tools 53 and 54, a skip instruction button 55 for instructing that a subsequent imaging process (e.g., the main imaging process) be skipped, a parameter adjusting button 56 to instruct that various types of parameters included in the image taking condition or the reconstruction condition be adjusted, an execution instruction button 57 to instruct that the subsequent imaging process be executed.

For example, when determining that the main imaging process is to be skipped as a result of viewing and studying the high dose equivalent image 50 and the assistance information 52 being displayed, the user is able to skip the main imaging process included in the workflow, by pressing the skip instruction button 55. As another example, when determining that the image taking condition or the imaged range needs to be changed as a result of viewing and studying the high dose equivalent image 50 and the assistance information 52 being displayed or when being presented with a recommended image taking condition by a separate piece of assistance information, the user is able to change the already-set imaged range and image taking condition by inputting information via the imaged range changing tools 53 and 54 and the parameter adjusting button 56. In yet another example, when determining that the main imaging process needs to be performed only on a specific range as a result of viewing and studying the high dose equivalent image 50 and the assistance information 52 being displayed, the user is able to set a range in which the image quality is insufficient relative to the high dose equivalent image, as an imaged range that needs to be set in the main imaging process, by using the imaged range changing tools 53 and 54.

When displaying the GUI for exercising control based on the assistance information by employing the controlling function 150a, the processing circuit 150 may display, with an emphasis, a button or the like corresponding to control recommended by the apparatus. For example, when the assistance information includes a judgment result stating that the “main imaging process does not need to be performed”, the skip instruction button 55 is displayed with an emphasis, as recommended control. The user is thus able to easily understand that the control corresponding to the button displayed with the emphasis is recommended.

Subsequently, by employing the controlling function 150a, the processing circuit 150 judges whether or not the user has instructed that the imaging protocol be changed (step S7). When the user has not instructed that the imaging protocol be revised (or changed) (step S7: No), the processing circuit 150 performs, by employing the controlling function 150a, a subsequent imaging process such as the main imaging process, without changing the imaging protocol set at step S1 (step S8).

On the contrary, when the user has instructed that the imaging protocol be changed (step S7: Yes), the processing circuit 150 changes the imaging protocol by employing the controlling function 150a, on the basis of the instruction input by the user (step S9). By employing the controlling function 150a, the processing circuit 150 controls operations of the X-ray CT apparatus 1 related to the imaging process, on the basis of the post-change imaging protocol (step S10).

For example, when the imaging protocol is changed at step S9 so as to “skip the main imaging process”, the processing circuit 150 skips the main imaging process by employing the controlling function 150a. In another example, when the imaging protocol is changed at step S9 by changing the “image taking condition”, the processing circuit 150 performs the main imaging process according to the post-change image taking condition, by employing the controlling function 150a.

Further, for example, it is also possible to spatially evaluate the image quality of the equivalent high dose image and to change the imaged range so that the main imaging process is to be performed only on a region that has not reached a reference level.

As explained above, the medical information processing apparatus 100 according to the present embodiment includes the data generating function 150d serving as the data generating unit, the information generating function 150e serving as the information generating unit, and the controlling function 150a serving as the controlling unit. For example, on the basis of the position determining imaging process serving as the first data obtained in the first imaging process (e.g., the position determining imaging process), the data generating function 150d is configured to generate the second data (e.g., the high dose equivalent projection data or the high dose equivalent reconstructed image data) equivalent to data obtained in an imaging process performed under an image taking condition different from that of the first imaging process. On the basis of the quality of the second data, the information generating function 150e is configured to generate the assistance information to assist the reviewing related to the second imaging process scheduled to be executed later than the first imaging process under the image taking condition different from that of the first imaging process. For example, the information generating function 150e is configured to perform the evaluating process using one of the high dose equivalent projection data and the high dose equivalent reconstructed image data and to generate the assistance information to assist the reviewing of the workflow including the imaging protocol, on the basis of the evaluating process. The controlling function 150a is configured to cause the display device 42 serving as a display unit to display the assistance information prior to the execution of the second imaging process.

In the evaluating process, one or more of the following are performed: calculating the evaluation value to evaluate the image quality of the high dose equivalent image and the like; detecting an abnormal site by using the high dose equivalent image; calculating the replacement level of the high dose equivalent image for the high dose image; and judging whether or not the main imaging process needs to be performed and whether or not the protocol of the main imaging process is suitable on the basis of the evaluation value, the detection result, and the replacement level. Further, as the assistance information based on the evaluating process, one or more of the following are generated: the evaluation information obtained in the evaluating process; the result of judging whether or not the main imaging process needs to be performed based on the evaluation information; the revision proposal for the protocol of the main imaging process based on the evaluation information; the replacement proposal for the protocol of the main imaging process based on the evaluation information; and the like.

By using the assistance information based on an objective index realized with the high dose equivalent image, the user is able to judge whether or not the already-set imaging protocol is suitable. Further, for example, when the high dose equivalent image has sufficient image quality to reach the level where a diagnosis process is possible, it is possible to omit the main imaging process or the high dose imaging process. On the contrary, for example, when the high dose equivalent image does not have sufficient image quality to reach the level where a diagnosis process is possible, it is possible to perform a high dose imaging process under a more optimal condition, by re-adjusting the parameters such as the image taking condition, the reconstruction condition, the imaged range, and/or the like on the basis of the assistance information. Further, from the high dose equivalent reconstructed image, when it is possible to find a lesion or the like that was not expected at the beginning of the medical examination and was not discoverable solely with a low dose image, it is possible to consider replacing or adding to the imaging protocol.

Accordingly, by using the objective index realized with the high dose equivalent image generated from the low dose data (the projection data or the reconstructed image data actually obtained by using the low dose in the position determining imaging process), it is possible to realize the method for determining whether or not the high dose imaging process is to be performed, re-adjusting the parameters, and replacing and/or adding to the imaging protocol, in an integrated and convenient manner. It is thus possible to establish the technique for reviewing the workflow related to the image diagnosis processes. As a result, it is possible to reduce radiation exposure, to shorten the time for the medical examinations, and to reduce burdens on the patients.

### First Modification Example

In the embodiments above, after the assistance information is displayed, only when the user explicitly changes the imaging protocol, the control related to the imaging process is exercised according to the post-change imaging protocol. Alternatively, the processing circuit 150 may be configured, by employing the controlling function 150a, to automatically skip the main imaging process or the like, on the basis of the judgment result included in the generated assistance information.

### Second Modification Example

In the embodiments above, the example was explained in which the high dose equivalent image or the like is used as the criterion for judging whether or not the main imaging process needs to be executed and whether or not the imaging protocol is suitable. Alternatively, for example, the processing circuit 150 may be configured, by employing the reconstruction processing function 150c, to generate a high dose equivalent subtraction image or the like, by using the high dose equivalent image or the like, as a non-contrast-enhanced image or the like of a subtraction process. Further, for example, the processing circuit 150 may be configured, by employing the reconstruction processing function 150c, to generate a high dose equivalent dual-energy image or the like, by using the high dose equivalent image or the like, as an image corresponding to one of the energy levels in a dual-energy imaging process or the like.

### Third Modification Example

Possible applications of the medical information processing apparatus 100 described in the above embodiments are not limited to image diagnosis processes using the X-ray CT apparatus 1. It is possible to apply the medical information processing apparatus 100 to image diagnosis processes using a PCT-CT system, an angio-CT system, and a magnetic resonance imaging apparatus.

For example, when the medical information processing apparatus 100 according to the above embodiments is applied to a PCT-CT system or an angio-CT system, it is possible, during an imaging process using an X-ray CT apparatus, to judge whether or not a main imaging process needs to be executed and whether or not an imaging protocol is suitable, on the basis of an objective index such as the high dose equivalent image or the like.

Further, when the medical information processing apparatus 100 according to the above embodiments is applied to a magnetic resonance imaging apparatus, for example, a sensitivity map or a locator obtained in a pre-scan (a preliminary imaging process) performed prior to a main imaging process is used for generating data (main imaging process equivalent data) equivalent to MR data or image data obtained in the main imaging process, as data corresponding to the high dose equivalent image of the above embodiments. It is possible to achieve the same advantageous effects as those of the above embodiments, by using the generated main imaging process equivalent data as an objective index.

### Fourth Modification Example

In the above embodiments, the example was explained in which the high dose equivalent image or the like is used as the criterion for judging whether or not the main imaging process needs to be executed and whether or not the imaging protocol is suitable. Alternatively, it is also possible to use the high dose equivalent data as a criterion for setting an image taking condition of the main imaging process or the like.

For example, in a lung cancer screening examination, low dose data is obtained by performing a position determining imaging process while using a low dose. By employing the data generating function 150d, the processing circuit 150 generates high dose equivalent data from the obtained low dose data. By employing the information generating function 150e, the processing circuit 150 detects an abnormal site by using the high dose equivalent data. Upon detection of an abnormal site, the processing circuit 150 performs, by employing the information generating function 150e, an evaluating process while using the high dose equivalent data, so as to generate, on the basis of an obtained evaluation result, assistance information including at least one of an image taking condition, an imaged range, and a reconstruction condition that is optimal for the main imaging process to be performed on the abnormal site.

### Fifth Modification Example

In the above embodiments, the example was explained in which the X-ray CT apparatus 1 is provided with the functions of the medical information processing apparatus 100. Alternatively, it is also acceptable to realize the medical information processing apparatus 100 capable of communicating with the X-ray CT apparatus 1, by using a medical workstation, a personal computer, or the like. The medical information processing apparatus 100 according to the first modification example structured in this manner is configured, for example, to receive data obtained by the X-ray CT apparatus 1 in a real-time manner and to perform the evaluating process and the assistance information generating process described above, by using the received data. The generated assistance information is displayed on the display device 42 or a monitor of the X-ray CT apparatus 1 in a real-time manner.

### Sixth Modification Example

To evaluate the quality of raw data, the raw data does not necessarily have to be directly evaluated. For example, it is possible to indirectly evaluate raw data, by evaluating the quality of image data reconstructed while using the raw data. Further, to evaluate the quality of image data, the image data does not necessarily have to be directly evaluated. For example, it is possible to indirectly evaluate image data, by evaluating the quality of raw data on which the image data is based.

According to at least one aspect of the embodiments described above, it is possible to establish the technique for reviewing the workflow related to the image diagnosis processes, by using the objective index.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising a processing circuit configured:
to generate, on a basis of first data obtained in a first imaging process, second data equivalent to data obtained in an imaging process performed under an image taking condition different from that of the first imaging process;
to generate, on a basis of quality of the second data, assistance information to assist reviewing related to a second imaging process scheduled to be executed later than the first imaging process under an image taking condition different from that of the first imaging process; and
to cause a display circuit to display the assistance information prior to the execution of the second imaging process.

2. The medical information processing apparatus according to claim 1, wherein the processing circuit causes the display circuit to display at least one button related to controlling the second imaging process, so as to be kept in association with the assistance information.

3. The medical information processing apparatus according to claim 1, wherein, by using the second data, the processing circuit generates the assistance information including at least one of: information related to whether or not the second imaging process needs to be executed; information related to whether or not a protocol of the second imaging process is suitable; information related to revising a protocol of the second imaging process; and information related to the quality.

4. The medical information processing apparatus according to claim 1, wherein
the first imaging process is a low radiation dose imaging process using an X-ray computed tomography apparatus, and
the second imaging process is a high radiation dose imaging process using an X-ray computed tomography apparatus.

5. The medical information processing apparatus according to claim 1, wherein
the first imaging process is one of a preliminary imaging process and a main imaging process that uses a magnetic resonance imaging apparatus, and
the second imaging process is a main imaging process that uses a magnetic resonance imaging apparatus.

6. The medical information processing apparatus according to claim 1, wherein the processing circuit exercises control related to the second imaging process, on a basis of the assistance information.

7. The medical information processing apparatus according to claim 1, wherein the processing circuit generates at least one of a subtraction image and a dual-energy image, by using the second data.

8. The medical information processing apparatus according to claim 1, wherein, on a basis of the quality, the processing circuit generates the assistance information including at least one of an image taking condition, an imaged range, and an image reconstruction condition related to a main imaging process.

9. A medical image diagnosis apparatus comprising:
a data generating unit configured, on a basis of first data obtained in a first imaging process, to generate second data equivalent to data obtained in an imaging process performed under an image taking condition different from that of the first imaging process;
an information generating unit configured, on a basis of quality of the second data, to generate assistance information to assist reviewing related to a second imaging process scheduled to be executed later than the first imaging process under an image taking condition different from that of the first imaging process; and
a controlling unit configured to cause a display circuit to display the assistance information prior to the execution of the second imaging process.

10. The medical image diagnosis apparatus according to claim 9, wherein the processing circuit causes the display circuit to display at least one button related to controlling the second imaging process, so as to be kept in association with the assistance information.

11. The medical image diagnosis apparatus according to claim 9, wherein, by using the second data, the processing circuit generates the assistance information including at least one of: information related to whether or not the second imaging process needs to be executed; information related to whether or not a protocol of the second imaging process is suitable; information related to revising a protocol of the second imaging process; and information related to the quality.

12. The medical image diagnosis apparatus according to claim 9, wherein
the first imaging process is a low radiation dose imaging process using an X-ray computed tomography apparatus, and
the second imaging process is a high radiation dose imaging process using an X-ray computed tomography apparatus.

13. The medical image diagnosis apparatus according to claim 9, wherein
the first imaging process is one of a preliminary imaging process and a main imaging process that uses a magnetic resonance imaging apparatus, and
the second imaging process is a main imaging process that uses a magnetic resonance imaging apparatus.

14. The medical image diagnosis apparatus according to claim 9, wherein the processing circuit exercises control related to the second imaging process, on a basis of the assistance information.

15. The medical image diagnosis apparatus according to claim 9, wherein the processing circuit generates at least one of a subtraction image and a dual-energy image, by using the second data.

16. The medical image diagnosis apparatus according to claim 9, wherein, on a basis of the quality, the processing circuit generates the assistance information including at least one of an image taking condition, an imaged range, and an image reconstruction condition related to a main imaging process.

17. A medical information processing method comprising:
generating, on a basis of first data obtained in a first imaging process, second data equivalent to data obtained in an imaging process performed under an image taking condition different from that of the first imaging process;
generating, on a basis of quality of the second data, assistance information to assist reviewing related to a second imaging process scheduled to be executed later than the first imaging process under an image taking condition different from that of the first imaging process; and
causing a display circuit to display the assistance information prior to the execution of the second imaging process.
